(19) United States
(12) Patent Application Publication
(10) Pub. No.: US 2022/0257620 A1
(43) Pub. Date: Aug. 18, 2022

(54) USE OF CERTAIN PHOSPHATIDYLCHOLINES CONTAINING LONG CHAIN POLYUNSATURATED FATTY ACIDS AS NEUROPROTECTIVE AGENTS

(71) Applicant: KANNALIFE SCIENCES, INC., Doylestown, PA (US)

(72) Inventors: Douglas E. BRENNEMAN, North Wales, PA (US); William A. KINNEY, Newtown, PA (US); Jason CLEMENT, Doylestown, PA (US); Dean PETKANAS, Lloyd Harbor, NY (US)

(21) Appl. No.: 17/622,861

(22) PCT Filed: Jun. 26, 2020
(86) PCT No.: PCT/US20/39860

§ 371 Date: Dec. 26, 2021

Related U.S. Application Data
(60) Provisional application No. 62/866,938, filed on Jun. 26, 2019.

(51) Int. Cl. A61K 31/685; A61K 36/185; A61K 31/05; A61P 25/28

(52) U.S. Cl. CPC A61K 31/685; A61P 25/28; A61K 31/05; A61K 36/185

(57) ABSTRACT

The invention relates to compositions containing certain phosphatidylcholines containing long chain polyunsaturated fatty acids in the sn-1 position or both the sn-1 and sn-2 positions as a neuroprotective agent, and further to treating or preventing oxidative stress in a neuronal tissue with such compositions.

### CROSS-REFERENCE TO RELATED APPLICATIONS

This application claims the benefit of U.S. Provisional Patent App. No. 62/866,938, filed Jun. 26, 2019, the content of which is incorporated by reference herein in its entirety.

### FIELD OF INVENTION

The invention relates to certain phosphatidylcholines containing long chain polyunsaturated fatty acids as a neuroprotective agent and to compositions and methods comprising the same.

### BACKGROUND OF THE INVENTION

An emerging concept is that neuroprotection by prevention of free radical mediated stress and oxidative stress will prevent neural damage. Compounds are capable of acting as neuroprotective agents by blocking the damage caused by free radicals and oxidative stress. Free radical mediated stress and oxidative stress is also known to contribute to additional pathological conditions including, but not limited to epilepsy, neuropathic pain, chemotherapy-induced peripheral neuropathy, traumatic head injury, stroke, Chronic Traumatic Encephalopathy (CTE), Post Cardiac Arrest Hypoxic Ischemic Encephalopathy, Epileptic Encephalopathy, Down syndrome, Hepatic Encephalopathy and neurodegenerative diseases such as Parkinson's disease, Alzheimer's, Huntington's disease, and amyotrophic lateral sclerosis (ALS). Compounds capable of acting as neuroprotective agents will be useful for the treatment of epilepsy, neuropathic pain, chemotherapy-induced peripheral neuropathy, traumatic head injury, stroke, Chronic Traumatic Encephalopathy (CTE), Post Cardiac Arrest Hypoxic Ischemic Encephalopathy, Epileptic Encephalopathy, Down syndrome, Hepatic Encephalopathy and neurodegenerative diseases such as Parkinson's disease, Alzheimer's, Huntington's disease, and amyotrophic lateral sclerosis (ALS).

There is a long felt need for neuroprotective agents that are both disease-modifying and effective in treating patients. In their endeavor to discover such neuroprotective agents, the inventors surprisingly found that certain phosphatidylcholines containing long chain polyunsaturated fatty acids are neuroprotective.

Phosphatidylcholines (PCs) are a class of phospholipids that incorporate choline as a headgroup. They are found endogenously, for example as a component of biological cell membranes. PCs are also found in brain membranes and it is believed that the fatty acyl groups of PCs are released by phospholipase A2 and are metabolized to various bioactive lipids as mediators of cell signaling. (Piomelli et al, 2007).

Sugiura et al., J Lipid Res 50:1776-1788, 2009 discloses cell-specific distributions of phosphatidylcholines with polyunsaturated fatty acids identified in specific regions of the mouse hippocampus. The study employed MALDi-IMS (imaging mass spectrometry) to demonstrate that PC 16:0-18:1 was the most abundant PC species in the mouse hippocampus and reports other PCs species as: 16:0-16:0; 18:0-18:1; 16:0-22:6; 16:0-20:4; 18:0-22:6; 18:0-20:4; 18:1-20:4; and 18:1-22:6. Like in a previous study (Yamashita et al., 1997), polyunsaturated fatty acids (PUFAs) arachidonic acid (20:4) and docosahexaenoic acid (22:6) were stored in the sn-2 position of the PC. Endogenous PCs's fatty acyl groups at the sn-1 position were all saturated or monounsaturated PCs 16:0, 18:0 or 18:1.

Previous studies by Charles Lieber demonstrate that dietary supplementation of dilinoleoylphosphatidylcholine (DLPC 18:2-18:2) could attenuate liver toxicity produced by chronic ethanol consumption. (Navder and Lieber, 2002). The concept advanced by the work of Lieber was that unsaturated phosphatidylcholines restored the structure of membranes and function of corresponding enzymes of the liver. (Lieber, 2005).

Additional studies with dietary supplementation of DLPC indicated that the mechanism of this protective effect on the liver involved inhibiting p38 MAPK in cultured hepatic stellate cells (Cao et al., 2002). The studies indicated that these liver effects were attributable to the antioxidant properties of DLPC and the inhibition of oxidative stress. These studies reported that the effects in hepatic cell cultures were observed at 10 μM. Kupffer cells from ethanol-fed rats were used to explore the mechanism of action of DLPC and indicated an association of a decrease in acetaldehyde-induced TNF-alpha in this system. (Cao et al., 2002, BBRC). The decrease in TNF-alpha by DLPC was believed to be mediated by blocking p38, ERK1/2 and NF-kappaβ activation.

Kafrawy et al., 1998 explored the cytotoxicity of PCs using PC liposomes with stearic acid in the sn-1 position and alpha-linolenic acid (18:3), arachidonic acid (20:4), or eicosapentaenoic acid (20:5) in the sn-2 position and PC with docosahexaenoic acid (22:6) in both the sn-1 and sn-2 position and found that the latter was unique in its ability to be incorporated into cell membranes to produce unique changes in the membrane structure incompatible with cell survival. The study proposes use of PC liposomes containing 22:6 as potential drug delivery vehicles to serve concomitantly as adjunct cancer therapy.

The invention addresses the need to prevent free radical mediated stress and oxidative stress, as well as to prevent the neural damage. The invention also addresses the long felt need for new treatments for and means of preventing diseases with free radical mediated stress and oxidative stress in their etiology, including, for example, epilepsy, neuropathic pain, chemotherapy-induced peripheral neuropathy, traumatic head injury, stroke, Chronic Traumatic Encephalopathy (CTE), Post Cardiac Arrest Hypoxic Ischemic Encephalopathy, Epileptic Encephalopathy, and neurodegenerative diseases such as Parkinson's disease, Alzheimer's, Huntington's disease, and amyotrophic lateral sclerosis (ALS).

All references cited herein are incorporated herein by reference in their entireties.

### SUMMARY OF THE INVENTION

Accordingly, a first aspect of the invention comprises a pharmaceutical composition comprising a neuroprotective amount of a phosphatidylcholine (PC), wherein the PC comprises a first polyunsaturated fatty acid (PUFA) in an sn-1 position thereof.

In certain embodiments, the PC further comprises a second PUFA in an sn-2 position thereof.

In certain embodiments, the first PUFA and the second PUFA comprise a fatty acyl chain with at least 16 carbons.

In certain embodiments, the first PUFA and the second PUFA comprise a fatty acyl chain with 18 to 24 carbons.

In certain embodiments, the first PUFA and the second PUFA are the same.

In certain embodiments, the first PUFA and the second PUFA are different.

In certain embodiments, the first PUFA is a member selected from the group consisting of 18:2ω6, 18:3ω3, 20:4ω6, and 22:6ω3.

In certain embodiments, the second PUFA is a member selected from the group consisting of 18:2ω6, 18:3ω3, 20:4ω6, and 22:6ω3.

In certain embodiments, the first PUFA is 20:4 and a fatty acyl chain in the sn-2 position is 16:0, 18:0 or 18:1.

In certain embodiments, the first PUFA is 20:4ω6.

In certain embodiments, the PC is a component of a natural extract.

In certain embodiments, the natural extract is an extract of Humulus lupulus.

In certain embodiments, the natural extract comprises at least one of the following PC species:

| | Chemical | Observed (m/z) | Calculated (m/z) |
|---|---|---|---|
| PC species | composition | [M + H]⁺ | [M + H]⁺ |
| PC 18:3-18:2 | C₄₄H₇₈NO₈P | 780.5540 | 780.5543 |
| PC 18:2-18:3 | C₄₄H₇₈NO₈P | 780.5540 | 780.5543 |
| PC 18:3-18:1 | C₄₄H₈₂NO₈P | 784.5857 | 784.5856 |
| PC 20:4-16:0 | C₄₄H₈₀NO₈P | 782.5704 | 782.5699 |

alone or in combination in an amount greater than 50% of the natural extract.

In certain embodiments, the natural extract is soy lecithin.

In certain embodiments, the soy lecithin comprises phosphatidylcholine with a polyunsaturation of the fatty acyl composition of at least 63%.

In certain embodiments, the pharmaceutical composition further comprises an excipient.

In certain embodiments, the pharmaceutical composition further comprises an additional active agent in addition to the PC.

In certain embodiments, the additional active agent is cannabidiol (CBD).

In certain embodiments, the PC is a membrane component of a liposome.

In certain embodiments, the pharmaceutical composition is for use in the treatment or prevention of oxidative stress in a neuronal tissue.

A second aspect of the invention comprises a method of treating or preventing oxidative stress in a neuronal tissue, said method comprising administering to a subject in need thereof the pharmaceutical composition of any preceding claim.

In certain embodiments of the method, the neuronal tissue comprises tissues and cells of the central nervous system.

Each of the first and second aspect of the invention encompasses any and all combinations of the features individually set forth in this Summary of the Invention.

### BRIEF DESCRIPTION OF THE DRAWINGS

FIG. 1 is a schematic diagram of the chromatographic fractionation of Humulus lupulus lipid extract.

FIG. 2 is a UV Chromatograph for silica gel purification of crude hexane fraction. Detection at 254 nm and 280 nm.

FIG. 3 shows the neuronal viability assay results for silica gel chromatography fractions.

FIG. 4 shows the cell death assay for silica gel chromatography fractions, with fraction 70 preventing cell death at 10⁵-fold dilution.

FIG. 5 shows the dose-response data for silica gel fraction 70, with protective effects on neuronal viability down to 10⁷-fold dilution.

FIG. 6 shows the dose-response data for silica gel fraction 70, with reduction in cell death down to 10⁷-fold dilution.

FIG. 7 shows the preparative C₁₈ HPLC trace for purification of pool of fractions from silica gel chromatography. Detection at 220 nm and 254 nm.

FIG. 8 shows the neuroprotective effects of fractions from preparative Cis HPLC chromatography.

FIG. 9 shows the reduction in cell death from treatment with fractions from preparative C₁₈ HPLC chromatograph.

FIG. 10 shows the dose-response testing of C₁₈ HPLC fraction 28, with neuroprotective effects down to 10⁷-fold dilution.

FIG. 11 shows the dose-response data for C₁₈ HPLC fraction 28, with reduction in cell death down to 10⁷-fold dilution.

FIG. 12 shows the analytical C₁₈ HPLC trace for purification of preparative Cis HPLC fraction 28.

FIG. 13 shows the protection of neuronal viability by analytical C₁₈ HPLC fractions derived from preparative C₁₈ HPLC fraction 28. Analytical C₁₈ HPLC fractions 9 and 10 possessed most of the neuroprotective effects of the parent chromatography fraction.

FIG. 14 shows the reduction of neuronal cell death from analytical C₁₈ HPLC fractions derived from preparative C₁₈ HPLC fraction 28.

FIG. 15 shows the dose-response testing of the protection of neuronal viability from treatment with analytical C₁₈ HPLC fraction 9.

FIG. 16 shows the dose-response testing of the reduction of cell death from treatment with analytical C₁₈ HPLC fraction 9.

FIG. 17 shows the dose-response testing of the protection of neuronal viability from treatment with analytical C₁₈ HPLC fraction 10.

FIG. 18 shows the dose-response testing of the reduction of cell death from treatment with analytical C₁₈ HPLC fraction 10.

FIG. 19 shows the dose-response testing of the protection of neuronal viability from treatment with analytical C₁₈ HPLC fraction 8.

FIG. 20 shows the dose-response testing of the reduction of cell death from treatment with analytical C₁₈ HPLC fraction 8.

FIG. 21 shows selective ion monitoring HPLC-MS traces for analytic C₁₈ HPLC fractions 8-11. SIM performed at 782.5704, corresponding to [M+H]⁺ (C₄₄H₈₁NO₈P)

FIG. 22 shows the fractionation tree for stem Humulus lupulus stem material. Activity was found only in a pool of Humulus lupulus stem preparative C₁₈ HPLC fractions 18-21.

FIG. 23 shows the improvement in neuronal viability from treatment with sample 54, which is the pool of Humulus lupulus stem preparative C₁₈ HPLC fractions 18-21.

FIG. 24 shows the reduction in cell death from treatment with sample 54, which is the pool of Humulus lupulus stem preparative C₁₈ HPLC fractions 18-21. All samples were tested at 10⁵-fold dilution relative to the original hexane extract of the H. lupulus stems.

FIG. 25 shows selective ion monitoring from HPLC-MS revealing the presence of phosphatidylcholine lipid esters in the neuroprotective pool of Humulus lupulus stem fractions 18-21.

FIG. 26 shows the EC50 for neuroprotection from ethanol toxicity of phosphatidylcholine with di-arachidonic acid in neuronal viability assay.

FIG. 27 shows the effect of phosphatidylcholine with di-arachidonic acid in cell death assay.

### DETAILED DESCRIPTION OF PREFERRED EMBODIMENTS OF THE INVENTION

The invention provides a method of treating or preventing oxidative stress in neuronal tissue with PCs, and preferably with PCs containing long chain polyunsaturated fatty acids (PUFAs) and to compositions comprising the same.

The PCs of the disclosure are capable of treating and preventing diseases associated with free radical mediated stress and oxidative stress in neuronal tissue including, for example, Parkinson's disease, Alzheimer's, Huntington's disease, traumatic head injury, stroke, epilepsy, neuropathic pain, chemotherapy-induced peripheral neuropathy, traumatic head injury, stroke, Chronic Traumatic Encephalopathy (CTE), Post Cardiac Arrest Hypoxic Ischemic Encephalopathy, and Epileptic Encephalopathy. It has been discovered that prevention of free radical mediated stress and oxidative stress will prevent damage and death of neuronal tissue, as well as prevent cognitive impairment, learning deficits, and memory impairment associated with damage and death of neuronal tissue. Without wishing to be limited by theory, it is believed that the PCs of the disclosure can ameliorate, abate, or otherwise cause to be controlled, diseases associated free radical mediated stress and oxidative stress. Diseases associated with free radical mediated stress and oxidative stress include, but are not limited to hepatic encephalopathy, epilepsy, neuropathic pain, chemotherapy-induced peripheral neuropathy, traumatic head injury, stroke, Chronic Traumatic Encephalopathy (CTE), Post Cardiac Arrest Hypoxic Ischemic Encephalopathy, Epileptic Encephalopathy, and neurodegenerative diseases such as Parkinson's disease, Alzheimer's, Huntington's disease, and amyotrophic lateral sclerosis (ALS).

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from a group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

The terms “treat” and “treating” and “treatment” as used herein, refer to partially or completely alleviating, inhibiting, ameliorating and/or relieving a condition from which a patient is suspected to suffer.

The terms “prevent” and “preventing” and “prevention” as used herein, refer to partially or completely alleviating, inhibiting, ameliorating and/or relieving a condition from which a patient is suspected to suffer or may suffer, such as from adverse drug side-effects produced by free radicals.

As used herein, “therapeutically effective” and “effective dose” refer to a substance or an amount that elicits a desirable biological activity or effect.

As used herein, the term “neuroprotection” shall mean the protecting of neurons in the brain, central nervous system or peripheral nervous system from death and/or damage. Preferably, the neurons are protected from death or damage caused by oxidative stress.

As used herein, the term “neuroprotective agent” shall mean a compound that provides neuroprotection.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the invention.

As used herein, the term “extract” refers to a composition derived from a source.

It has surprisingly been discovered that certain PCs containing PUFAs exhibit neuroprotective effect on neuronal tissues subjected to oxidative stress. The PCs of the disclosure demonstrate neuroprotective effect in maintaining neuronal viability and preventing neuronal cell death in hippocampal cultures subjected to ethanol toxicity. The PCs of the disclosure demonstrate neuroprotective potency by preventing neurotoxicity at concentrations ranging from 0.5 μM to 1 nM as shown in viability and cell death assays in hippocampal cultures co-treated with 30 mM ethanol to produce oxidative stress.

The method of treatment with PCs of the invention preferably comprises administering PCs with a PUFA in the sn-1 position. In certain embodiments, the PCs of the invention further comprise a PUFA in the sn-2 position. The PUFAs in the sn-1 and/or sn-2 of the PCs of the invention may be the same or different. Most preferably, the PUFAs in the sn-1 and sn-2 are the same.

Preferably, the PUFAs in the PCs of the invention comprise long chain PUFAs with at least 18 carbons. Preferably, the PUFAs comprise a fatty acyl chain with 18 to 36 carbons, more preferably from 18-24 carbons, and most preferably from 18-22 carbons.

Preferably, the PCs of the invention comprise omega-3 (ω3) or omega-6 (ω6) PUFAs. Most preferably, the PCs of the invention comprise omega-6 PUFAs.

Preferably, the PCs of the invention comprise PUFAs such as linoleic acid (18:2), linolenic acid (18:3), arachidonic acid (20:4), and docosahexaenoic acid (22:6ω3). More preferably, the PUFAs are 18:2ω6, 18:3ω3, 20:4ω6 and 22:6ω3 either in the sn-1 or both the sn-1 and sn-2 positions.

Preferably, the PC of the invention is a 1,2-dilinoleoyl-phosphatidylcholine (PC 18:2-18:2), 1,2-dilinolenoyl-phosphatidylcholine (PC 18:3-18:3), a 1,2-diarachidonyl-phosphatidylcholine (PC 20:4-20:4), a 1-linolenoyl-2-linoleoyl-phosphatidylcholine (PC 18:3-18:2), a 1-linoleoyl-2-linolenoyl-phosphatidylcholine (PC 18:2-18:3), or a 1-linolenoyl-2-oleoyl-phosphatidylcholine (PC 18:3-18:1). Most preferably, the PC is 1,2-diarachidonyl-phosphatidylcholine (PC 20:4-20:4) or a 1-arachidonyl-2-palmitoyl-phosphatidylcholine (PC 20:4-16:0).

The sn-position of the fatty acyl groups on PCs has been surprisingly found to be important to the neuroprotective effect of the PCs on neuronal tissue. The inclusion of a saturated fatty acyl group in the sn-1 position is found to dramatically decrease the potency of the PC by 3000-fold in comparison to the PC with 20:4 in both the sn-1 and sn-2 positions. As known to the inventors at this time, PUFAs are mainly found in the sn-2 position of endogenous PCs in mammalian nervous system and not in the sn-1 position. Rather, saturated or monounsaturated fatty acyl groups are found in the sn-1 position, such as 16:0, 18:0 or 18:1.

The potency of PCs of the disclosure in neuroprotective activity or effect is also surprising compared to other reports of PC related biological activity. EC50 and IC50 values of 0.5 pM to 1 nM have been achieved using PCs of the invention as neuroprotective agents, whereas DLPC (18:2), for example, has been reported to exhibit other biological activity at concentrations of at least 10 μM.

The invention also relates to compositions or formulations which comprise the PCs of the invention to treat and prevent oxidative stress in neuronal tissues, particularly neuronal tissues of the CNS.

In general, compositions of the invention comprise an effective amount of one or more PCs of the invention to treat or prevent oxidative stress in a neuronal tissue and/or treat or prevent a neurological disorder characterized by oxidative stress of neuronal tissues.

The composition may further comprise at least one excipient. For the purposes of the invention the term “excipient” and “carrier” are used interchangeably throughout the description of the invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being inert filler, or an excipient as used herein may be part of a pH stabilizing system or coating to insure delivery of the ingredients safely to the stomach. Examples of such excipients are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes.

The composition is preferably pharmaceutically acceptable. As used herein, “pharmaceutically acceptable” refers to a substance that is acceptable for use in pharmaceutical applications from a toxicological perspective. Pharmaceutically acceptable ingredients of the composition additional to the PC should not adversely interact with the PC or each other.

Supplementary active ingredients can also be incorporated into the compositions of the invention.

As phospholipids are well-established excipients in pharmaceuticals, the PCs of the disclosure may play a dual role in formulations of the invention. In addition to providing neuroprotective activity, the PCs of the disclosure may also be used as a vehicle to deliver or improve the bioavailability of additional active agents, particularly other neuroprotective agents. The PCs may thus additionally serve as a vehicle for an adjunct, combination, or synergistic therapy of PCs of the invention with an additional active agent.

The compositions of the present invention may comprise PCs of the disclosure in combination with additional active agents. The additional active agent may be, for example, other active agents used to treat neurological disorders. Non-limiting examples of the additional active agent are: cannabidiol (CBD), KLS-13019, levodopa, bromocriptine, pergolide, pramipexole, ropinirole, selegiline, benztropine, trihexyphenidyl, amitriptyline, amoxapine, clomipramine, desipramine, doxepin, imipramine, maprotiline, nortriptyline, protriptyline, amantadine, trimipramine, diphenhydramine, haloperidol, chiorpromazine, olanzapine, benzodiazepines, paroxetine, venflaxin, lithium, valproate, carbamazepine, fluoxetine, paroxetine, sertraline, escitalopram, citalopram, fluvosamine, citalopram, atomoxetine, memantine, rivastigmine, donepezil, gabapentin, pregabalin and the like. Most preferably, the additional active agent is CBD or KLS-13019.

The PCs of the disclosure may be used in formulations including but not limited to softgels, fat emulsion, mixed micelles, suspensions and liposomal preparation by methods known in the art. In certain embodiments, the PCs may be a component of liposomes, micelles, mixed micelles, nanoparticles, solid lipid nanoparticles, cubosomes and the like. The formulation and compositions may in certain embodiments take advantage of the PCs amphiphilic character, such as the ability to form liposomes, micelle, and bilayers in solution with hydrophilic heads facing the outside environment and hydrophobic tails facing inwards.

The composition of PCs of the invention may be formulated for administration by any route, for example, oral and parenteral.

When administered for the treatment or inhibition of a particular disease state or disorder, it is understood that an effective dosage can vary depending upon the particular PC or composition of PCs utilized, the mode of administration, and severity of the condition being treated, as well as the various physical factors related to the individual being treated. In therapeutic applications, a PC or composition of PCs of the invention can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

Compositions and PCs of the invention for treatment and prevention of oxidative stress can be prepared from natural products, commercially available starting materials, compounds known in the literature, or readily prepared intermediates, by employing standard extraction, chromatographic and synthetic methods and procedures known to those skilled in the art. Extraction and chromatographic fractionation from natural products and standard synthetic methods and procedures for the preparation of compositions comprising natural and synthetic PCs, including synthetic analogs of natural PCs, for use in the invention can be readily obtained from the relevant scientific literature or from standard textbooks in the field. This includes, for example, enzyme modification of natural PCs to modify the acyl group in the sn-1 and sn-2 positions with enzymes known in the art.

Natural products or sources for the PCs for use in the invention may come from a variety of sources, such as, e.g., egg yolk, soybean, rapeseed, sunflower seed, flax seed, wheat germ, and the like. Preferably, the natural products or sources comprise a high concentration of PCs of the invention. The PCs for use in the invention may be isolated and purified from the natural products or sources or maybe utilized as a component of an extract of the natural product or source. The extract from natural products or sources preferably comprises PCs in a weight percentage of more than about 20%, more than about 45%, more than about 68%, more than about 75%, or more than 98%. The extract from natural products or sources preferably comprises PCs with a polyunsaturation of the fatty acyl group of at least 20%, at least 45%, at least 63%, and more preferably at least 75%. The extract from natural products or sources preferably comprises 30-95%, more preferably 40-85%, preferably 50-75%, and most preferably 55-60% of PCs of the invention. Preferably, the natural source is soybean or Humulus lupulus. Preferably the extract is soy lecithin or an extract of Humulus lupulus. Most preferably, the extract is a highly purified fraction of Humulus lupulus comprising one or more of the PCs listed in Table 2.

The invention will be illustrated in more detail with reference to the following Examples, but it should be understood that the present invention is not deemed to be limited thereto.

### EXAMPLES

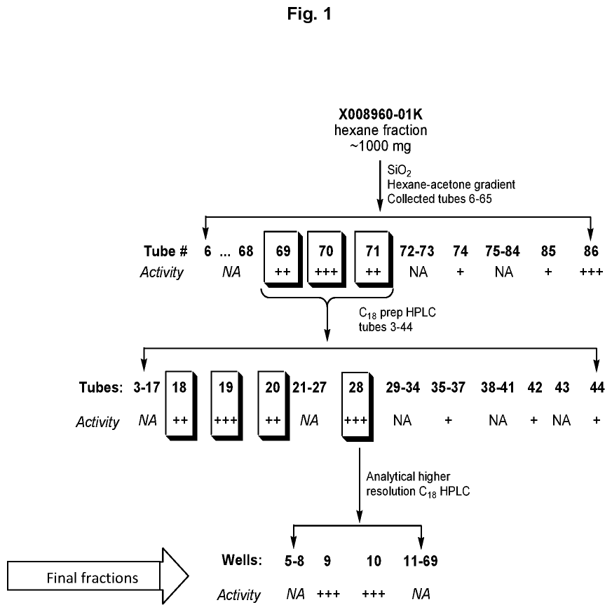

Examples 1-3 provides the chromatographic procedures used to identify and obtain neuroprotective compounds from Humulus lupulus. Fractions exhibiting high potency neuroprotection from oxidative stress in hippocampal cultures ware selected for further purification and testing. The chromatographic procedures are summarized in the fractionation tree shown in FIG. 1.

### Example 1: Silica Gel Chromatography Fractions

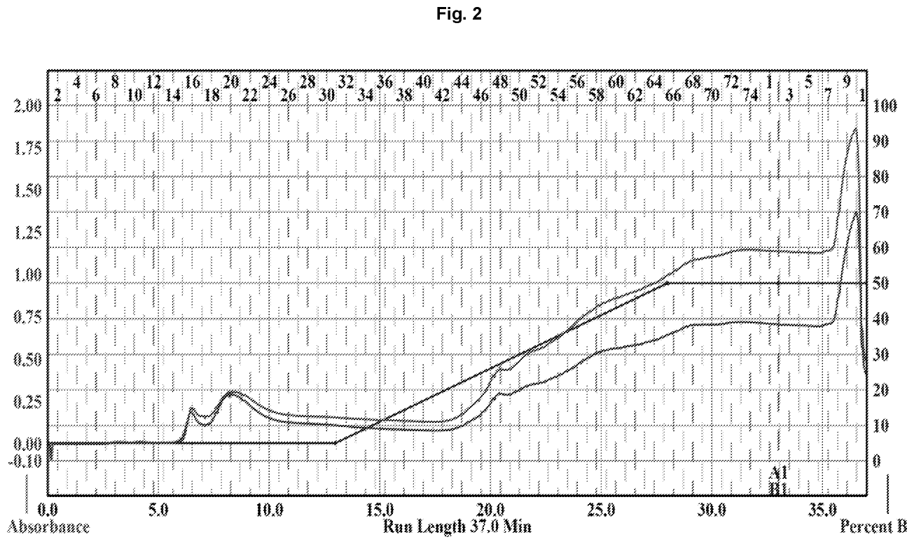

Aerial parts (leaves, stems, and fruit) of Humulus lupulus were extracted with methanol, and the extract was concentrated to dryness. This crude methanol extract (10.0 g) was then partitioned between hexane (200 mL) and 90% aqueous methanol (200 mL), by washing the aqueous methanol layer with hexane in two portions. The hexane washes were pooled and concentrated under vacuum to afford 1.52 g of hexane-soluble material. From this hexane fraction, 933 mg was taken and fractionated by silica gel open column chromatography (silica gel: Fisher #S826-212). The height of the column was 17.5 cm. The width of the column was about 2.4 cm. The column was eluted with a binary gradient of hexane and acetone using a CombiFlash Rf system. The gradient was as follows: 5% acetone, 13 minutes; 5%-50% acetone, linear gradient for 15 minutes; hold at 50% acetone for 5 minutes; then the column was flushed with 100% methanol for 5 minutes; 23 mL/min flow rate, 10 mL/fraction. A total of 86 fractions were collected. The chromatogram for this purification is shown in FIG. 2.

### Example 2: Preparative C₁₈ HPLC Fractions

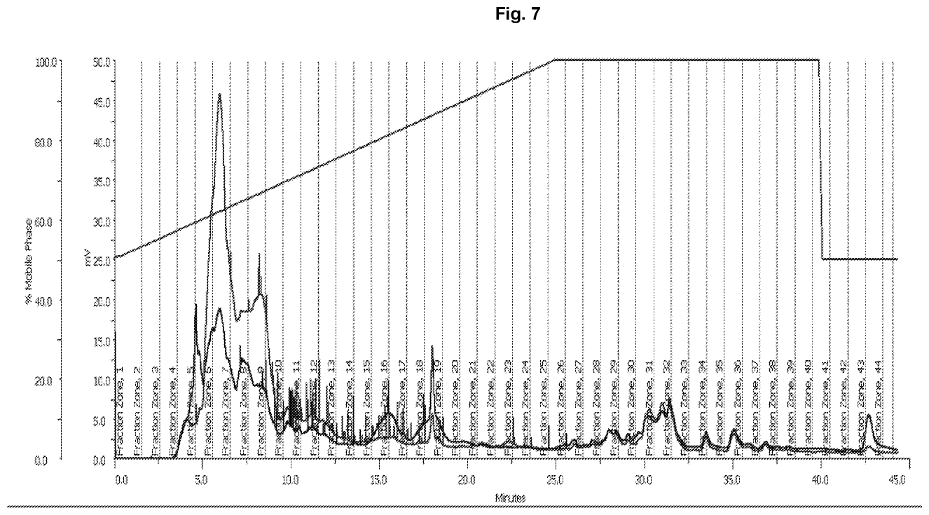

Silica gel chromatography fractions 69, 70, and 71 were pooled and selected for further purification. The pool of silica gel fractions 69-71 was fractionated using an Agilent Zorbax PrepHT XDB C₁₈ column (21.2 mm×250 mm, 7 m particle size, 100 Å pore size, 10 mL/min) with a binary gradient of acetonitrile and water. The gradient was as follows: 50%-100% acetonitrile, 25 minutes; hold at 100% acetonitrile for 15 minutes, return to original conditions and hold for four minutes; 10 mL/fraction. This purification afforded 44 fractions. FIG. 7 is the HPLC UV trace at 220 nm and 254 nm.

### Example 3: Analytical C₁₈ HPLC Fractions

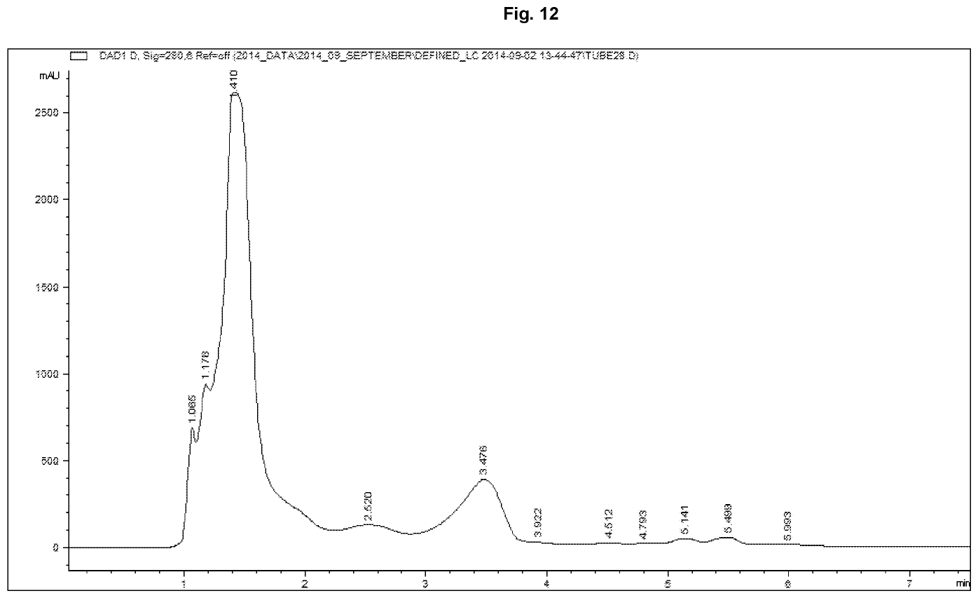

Fraction 28 from the preparative C₁₈ purification was further purified using an Agilent Eclipse Plus C18 column (4.6×100 mm, 3.5 m particle size). A binary, linear gradient of acetonitrile and water was used. The gradient was 70%-100% acetonitrile over 30 minutes. The sample was dissolved in 50 μL of methanol, and 48 μL was injected for the purification. The first 15 fractions were collected every 12 seconds, then 8 fractions were collected every 6 seconds, and finally 46 fractions were collected every 8 seconds for a total of 69 fractions collected over 9 minutes and 56 seconds. The HPLC-UV chromatography trace at 280 nm for the analytical C₁₈ purification of fraction 28 is shown in FIG. 12.

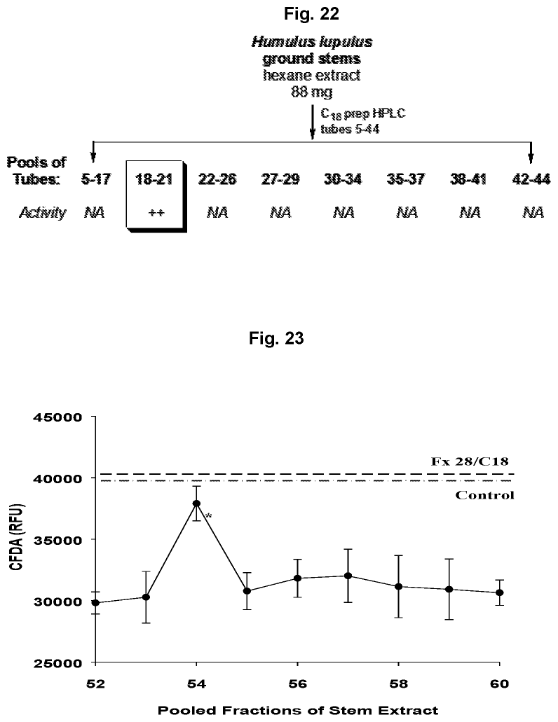

Example 4 provides the chromatographic procedures used to identify and obtain neuroprotective compounds from Humulus lupulus ground stem material. The chromatographic procedures are summarized in the fractionation tree shown in FIG. 22.

### Example 4: Fractions from H. lupulus Stems

An extraction was performed on stems of Humulus lupulus. Dried, ground stem material (195 g) was extracted with hexane (980 mL) by shaking overnight. The extraction was filtered through Whatman filter paper with a Buchner funnel and the filtrate was concentrated to dryness to yield 1.23 g of hexane extract. Of this stem extract, 88 mg was fractionated by the same HPLC method referenced in Example 2 to afford 44 fractions.

Example 5 provides formulation embodiments comprising natural sources of PCs of the invention and comparative natural source phospholipid compositions. The formulations were tested alone and with another active agent for neuroprotective activity.

Table 1: shows exemplary softgel formulations of natural compositions comprising PCs of the invention and comparative natural phospholipid compositions with an additional API. Without the additional API, the weight percentages of the other components of the formulations remain the same with the remaining 20% comprising an inactive substance.

| Formu- | | | Additional |
|---|---|---|---|
| lation | Compositions | PC source | API |
| A1 | 56% olive oil/8% lauroglycol | 8% Soy Lecithin | 20% |
| | FCC/8% vitamin E TPGS | non-GMP | |
| A2 | 56% olive oil/8% lauroglycol | 8% Egg Lecithin | 20% |
| | FCC/8% vitamin E TPGS | GMP | |
| A3 | 50% Maisine CC/22% Kolliphor | none | 20% |
| | ELP. 8% vitamin E TPGS | | |
| A4 | 56% olive oil/8% lauroglycol | 8% Soy Lecithin | 20% |
| | FCC/8% vitamin E TPGS | GMP | |

* Maisine CC: glyceryl monolinoleate
*Kolliphor ELP: polyoxyl 35 Castor Oil - surfactant.

### Procedures

The following procedures can be utilized in evaluating compounds and compositions as neuroprotective agents against ethanol toxicity.

Preparation of Humulus lupulus Fractions of Examples 1-4

The silica gel chromatography fractions of Example 1 were tested for neuroprotective activity in samples, either as pools or as individual fractions. The samples were all tested at a concentration equivalent to 100 ng/mL of the original crude extract, based on the amount of crude extract applied to the column. The preparative C₁₈ fractions of Example 2 were tested individually or in pools. The fractions were tested at a dilution level equivalent to 100 ng/mL of the crude extract. The analytical C₁₈ fractions of Example 3 were tested either individually or as pools at a dilution level equivalent to 100 ng/mL of the crude hexane fraction. The fractions pools from Humulus lupulus stems of Example 4 were tested at a dilution level equivalent to 100 ng/mL of the crude hexane extract of the stems of H. lupulus.

Source and Preparation of Commercially Available Phosphatidylcholines

All the commercial PCs included in this disclosure were obtained from Avanti Polar Lipids, Inc. (Alabaster, Ala.). All PCs were obtained in sealed ampoules either as a powder or in a chloroform solution at 25 mg/2.5 ml. For PCs that were in powder form, 25 mg samples of PC were dissolved in a solution of chloroform-methanol (2:1 v/v) to a final concentration of 25 mg in 2.5 ml. Once in solution, 0.1 mL containing 1 mg of each PC in solution was placed in a sterile glass vial for evaporation with a stream of Argon gas. Upon obtaining sample dryness from the organic solvent, each of the 1 mg samples were dissolved in dimethyl sulfoxide (DMSO) and then thoroughly mixed. The amount of DMSO was adjusted for each PC preparation to produce a 10 mM stock solution. The PC sample in DMSO was then mixed with 990 mL of Dulbecco's phosphate buffered saline (DPBS) obtained from Gibco (Grand Island, N.Y.) to produce a working stock dilution of 100 μM from which serial dilutions with DPBS were made to study the concentration effects of each PC. Log concentrations ranging from 0.1 μM (10⁻⁷ M to 0.01 μM (10⁻¹⁴M were prepared for testing. For the final testing, 10 ul of each of the concentration-based test solutions were put into 90 ul of culture medium for each of the culture wells to give a final testing concentration of PC. The final concentration range tested was from 0.01 μM to 0.1 μM. In no test did the amount of DMSO in the test exceed that of 0.1% to ensure no biological contribution of this solvent to the result. For each of the PC tested, at least six concentrations were used to determine the concentration-effect curve and the EC50s. The following PC samples were obtained from Avanti Polar Lipids along with the catalog number: 18:3 Cis (850395C), 20:4 Cis (850397C), 22:6 Cis (850400C), 18:1 Cis (850375P), 18:2 Cis (850385P), 18:0-18:2 (850468P), 16:0-18:2 (850458P), 18:0-18-1 (850467P). In addition to the PCs tested, two other phospholipids were also tested that contained fatty acyl groups of 18:2 including phosphatidylethanolamine (PE) and phosphatidic acid (PA). These samples from Avanti Polar Lipids were: 18:2 PE (850755C) and 18:2 PA (840885C).

Cell Cultures

All compounds and compositions were screened with dissociated hippocampal cultures derived from embryonic day 18 rats as the primary test system. With this preparation, primary neurons were used to test for toxicity as well as neuroprotection. In brief, hippocampal tissue was obtained commercially through Brain Bits (Springfield, Ill.) and cultures prepared as previously described (Brewer, 1995). The hippocampal neurons were plated at low density (10,000 cell/well) in a 96-well format and maintained in serum-free medium consisting of Neurobasal Medium supplemented with B27 and GlutaMAX (Gibco). Pre-coated poly-D-lysine coated plates will be used because of the adherence and survival of hippocampal neurons and glia on this matrix support.

Ethanol as a Toxic Agent

The testing for protection from oxidative stress was based on using ethanol as a source of cellular toxicity and the generation of reactive oxygen species that produces the toxicity. Dose response studies indicated that 30 mM ethanol produced a robust and reproducible toxicity as measured with both the CFDA and PI assays described below for hippocampal cultures. For all of the studies reported in this disclosure, 30 mM ethanol was used as the toxic agent that produced the decreases in neuronal viability and the increases in cell death in the hippocampal cultures. Although ethanol was used as the agent of choice for this disclosure, other toxic agents known to be relevant to neurological disease have also been tested in the hippocampal system including: hydrogen peroxide, ammonium acetate, glutamate and heavy metals. The effects of ethanol was intended to provide an example of an agent that's produces reactive oxygen species and oxidative stress in hippocampal cultures, but the effects of the protective actions of PCs should not be regarded as limiting to the effects of ethanol.

Vital Dyes Utilized

Carboxyfluorescein (CFDA) was used a vital stain for all neuroprotection studies. With the use of the CytoFluor fluorimeter, the CFDA assay was employed to assess the viability of neurons. CFDA is a dye that becomes fluorescent upon cell entry and cleavage by cytosolic esterases (Petroski and Geller, 1994). Neuronal specificity is obtained relative to astrocytes because the cleaved dye is extruded extracellularlly by glia with time, while dye in neurons remains intracellular. Previous experience with this assay showed a good correlation with neuronal cell counts stained immunocytochemically with neuron specific enolase antibodies, a reference marker for neuronal identity in complex cultures. To further asses the culture responses, a propidium iodide method was used as previously described (Sarafian et al., 2002) to measure the number of dead cells. Propidium iodide becomes fluorescent when binding to the DNA of dead cells.

Protection from Oxidative Stress that Produced Cell Death

Experimental details for the propidium iodide assay (Sarafian, T. A.; Kouyoumjian, S.; Tashkin, D.; Roth, M. D. Synergistic cytotoxicity of 9-tetrahydrocannabianol and butylated hydroxyanisole, Tox. Letters, 2002. 133, 171-179.) To test for protection from 30 mM ethanol, day 11 hippocampal cultures were given a complete change of medium containing 100 μl of Neurobasal medium with B27 (Gibco). After a complete change medium, the ethanol neuroprotection studies were started. In this disclosure, hippocampal cultures ranging from day 11 to day 18 were included in the study, as this developmental period in hippocampal cultures provided robust and reproducible responses to both ethanol toxicity and the protective effects of the PCs. A compound of the disclosure was added to the hippocampal cultures for a 5 hour test period in concentrations that ranged from 0.01 μM to 0.1 μM. Concurrent with the treatment of a compound of the disclosure, 30 mM ethanol was added for the 5 hour test period. At the conclusion of the test period, the cultures were tested for the amount of cell death by the propidium iodide method. Propidium iodide (PI) stock solution of 1 mg/ml (1.5 mM) was obtained from Sigma. The PI stock was diluted 1:30 in DPBS for a final working concentration of 50 μM. After removal of the growth medium, 50 μl of the 50 mM PI solution was added to cultures and allowed to incubate in the dark at room temperature for 15 min. The cultures were then assessed for fluorescence intensity at Ex536/Em590 nm in a CytoFluor fluorimeter. Results were expressed in relative fluorescent units and IC50's calculated from the dose response of the compounds and compositions of the disclosure. Each value is the mean of at least 8 determinations±the standard error completed in replicate tissue culture plates in at 96-well format. The PI assay was multiplexed with the CFDA assay described below so that within each well, both cell death and neuronal viability could be measured.

Neuroprotection from Oxidative Stress that Produced Neuronal Toxicity

Experimental details for the CFDA assay (Petroski, R. E.; Geller, H. M Selective labeling of embryonic neurons cultures on astrocyte monolayers with 5(6)-carboxyfluorescein diacetate (CFDA). J. Neurosci. Methods 1994, 52, 23-32.) To test for neuroprotection from 30 mM ethanol, day 11 hippocampal cultures were given a complete change of medium consisting of 100 μl of Neurobasal medium with B27 (Gibco). After the complete change in medium, the ethanol neuroprotection studies were started. The compound of the disclosure was added to the hippocampal cultures for a 5 hour test period in concentrations that ranged from 0.01 μM to 0.1 μM. Concurrent with the treatment of the compound of the disclosure, 30 mM ethanol was added for the 5 hour test period. At the conclusion of the test period, the cultures were tested for the amount of neuronal viability by the CFDA method. For the neuronal viability assay, 1 mg of 5,6-Carboxyfluorescein diacetate (CFDA) dye (Sigma) was dissolved in 100 ml of DPBS (Gibco:D-5780) and kept in the dark until added to the hippocampal cultures. After a complete change of medium, 100 μl CFDA dye solution was added for 15 min of incubation at 37 degrees in the dark. At the conclusion of the incubation period, the dye was removed from the cultures and washed once with 100 μl of DPBS. After removal of the first wash, a second wash of DPBS was added to the culture and then incubated for 30 min to allow the efflux of dye out of glia in the cultures. At the conclusion of the 30 min efflux period, the culture efflux medium was removed and 100 μl of 0.1% triton-X in water 100 was added to the cultures to before reading at Ex490/Em517 in a CytoFluor fluorimeter. Results were expressed in relative fluorescent units (RFU) and EC50's calculated from the dose response of the compound of the disclosure. Each value is the mean of at least 8 determinations±the standard error completed in replicate tissue culture plates in at 96-well format.

All EC50 and IC50 values were generated with the curve-fitting procedure provided by the four-parameter logistic analysis with in SigmaPlot 11.

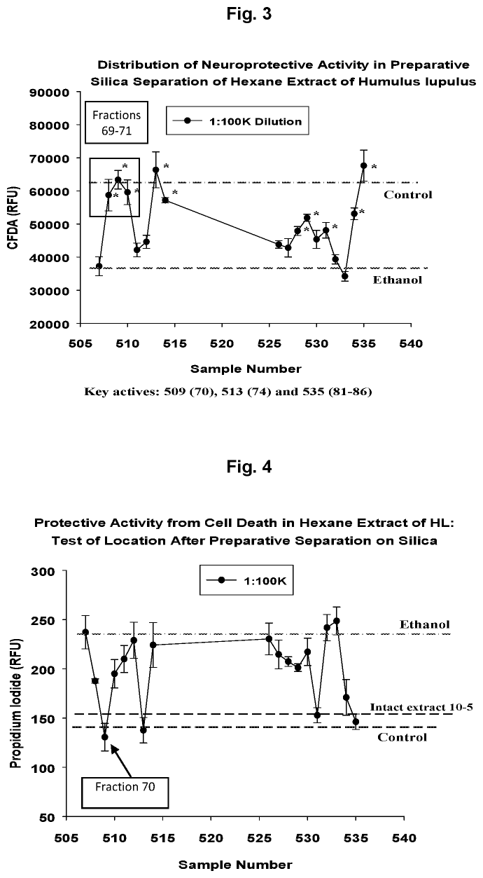

Results for representative compounds and compositions of Examples 1-4, according to the invention, are shown in FIGS. 3-6, 8-11, 13-20, and 23-24.

FIGS. 3-6 show the testing results of the silica gel chromatography fractions of Example 1. As shown in FIG. 3, samples 508, 509, and 510 (corresponding to chromatography fractions 69, 70, and 71) had neuroprotective activity, while samples 513 (fraction 74) and 535 (pool of fractions 81-86) also had neuroprotective activity.

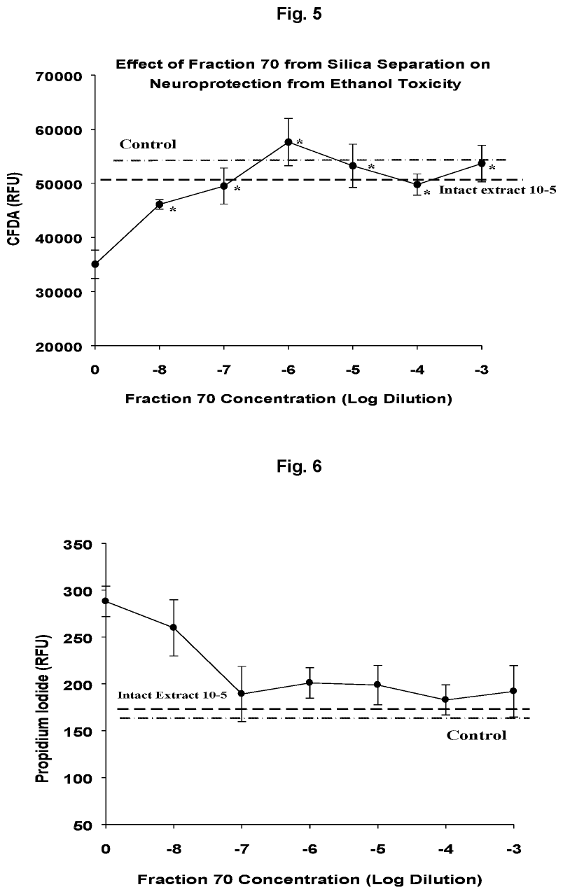

Additionally, samples 509, 513, and 535 also prevented cell death due to ethanol exposure, as shown in FIG. 4. Fraction 70 was examined further in dose-response experiments for neuroprotective activity. As shown in FIG. 5, fraction 70 maintained full neuroprotective activity down to a 10⁷-fold dilution, equivalent to 1 ng/mL of the crude extract. As displayed in FIG. 6, fraction 70 also maintained protection from cell death down to 10⁷-fold dilution, or equivalent to 1 ng/mL of the crude extract.

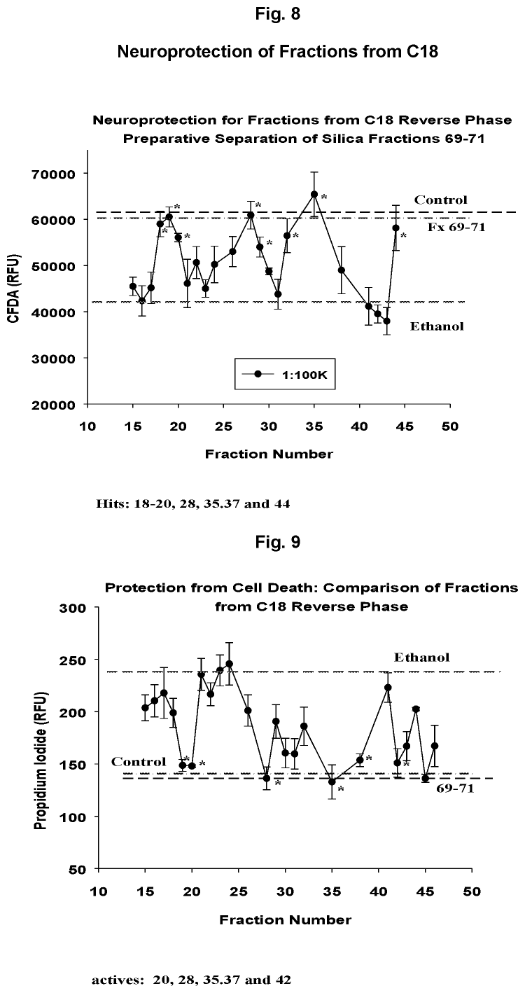

FIGS. 8-11 show the testing results of the preparative C₁₈ HPLC fractions of Example 2. As depicted in FIG. 8, enhancement of neuronal viability was detected in fractions 18-20, fraction 28, a pool of fractions 35-37, and fraction 44. Furthermore, preparative C₁₈ HPLC fractions 20, 28, the pool of fractions 35-37, and fraction 42 were found to prevent cell death at a concentration equivalent to 100 ng/mL of the crude extract, as shown in FIG. 9.

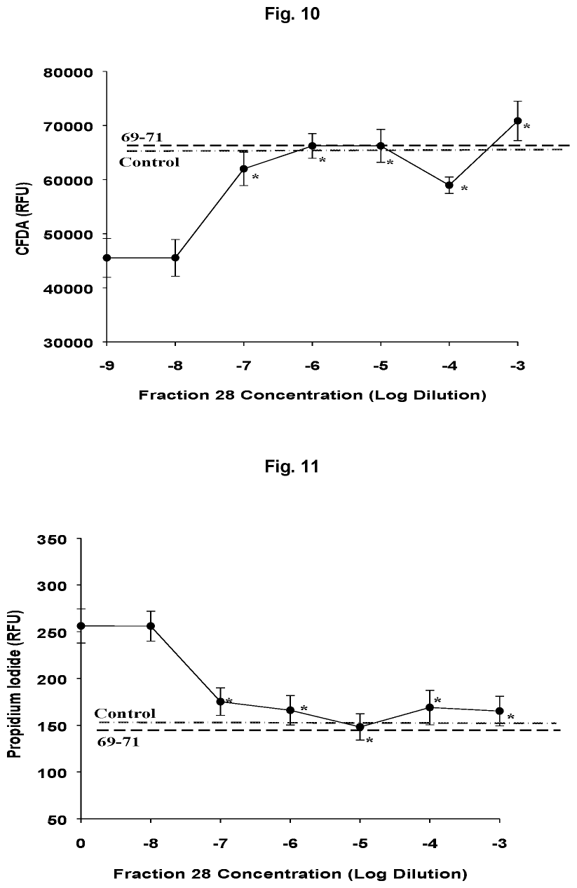

Fraction 28 was subjected to dose-response testing for neuroprotective activity. As shown in FIG. 10, fraction 28 was found to maintain full neuroprotective activity down to a 10⁷-fold dilution equivalent to 1 ng/mL of the crude extract. Fraction 28 was also found to prevent cell death at a 10⁷-fold dilution as well, as shown in FIG. 11.

Preparative C₁₈ chromatography fraction 28 had a mass of 0.6 mg that was purified from 933 mg of crude hexane fraction. For neuroprotection assay, fraction 28 was diluted to a concentration level equivalent to 10 mg/mL of crude hexane fraction based on the amounts loaded onto the C₁₈ HPLC column. If 0.6 mg of fraction 28 is equivalent to 933 mg of crude hexane fraction, then 1 ng of crude hexane fraction is equivalent to 0.64 pg of fraction 28. Therefore, fraction 28 was active down to a level of approximately 0.64 pg/mL.

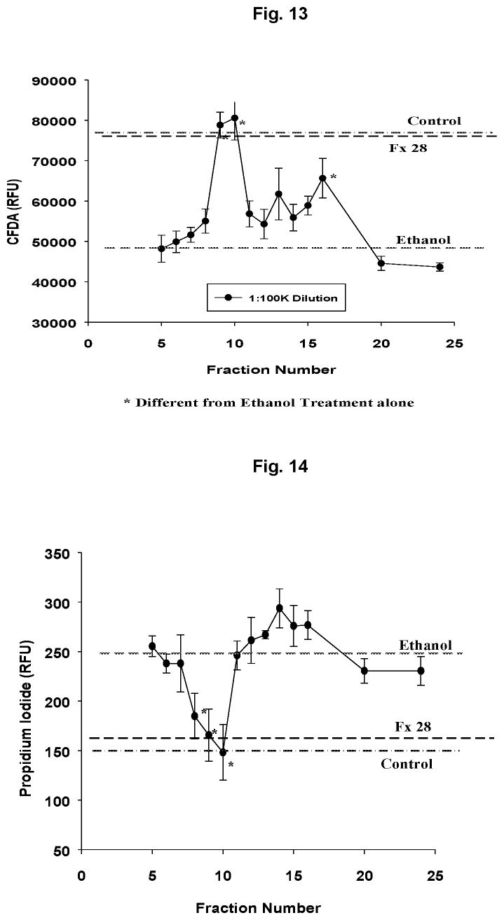

FIGS. 13-20 show the testing results of the analytical C₁₈ HPLC fractions of Example 3. From the analytical C₁₈ fractionation, the fractions were tested either individually or as pools. FIG. 13 shows the neuroprotective activity for the various analytical C₁₈ chromatography fractions. At a dilution level equivalent to 100 ng/mL of the crude hexane fraction, the highest activity was detected in fractions 9 and 10. Additionally, the fractions were tested for their ability to prevent cell death upon ethanol exposure, and fractions 9 and 10 were most effective at a concentration equivalent to 100 ng/mL of the crude hexane fraction (FIG. 14).

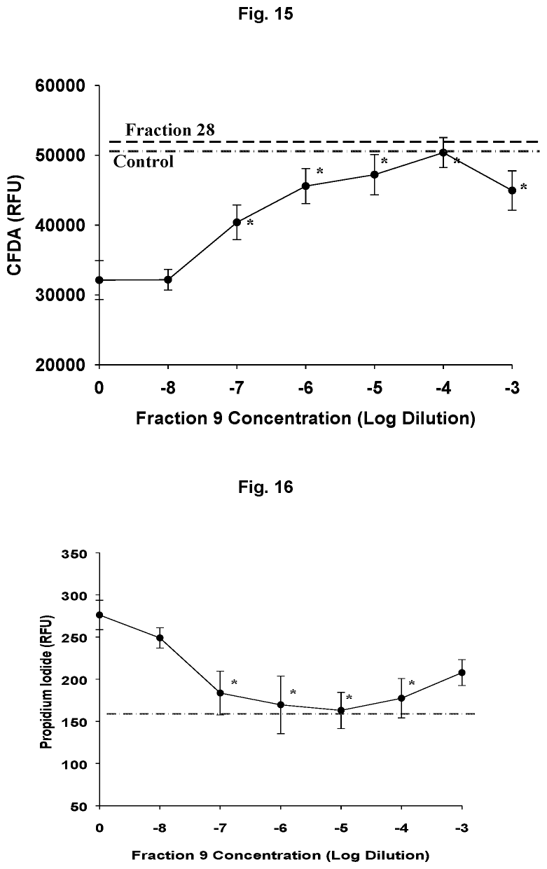

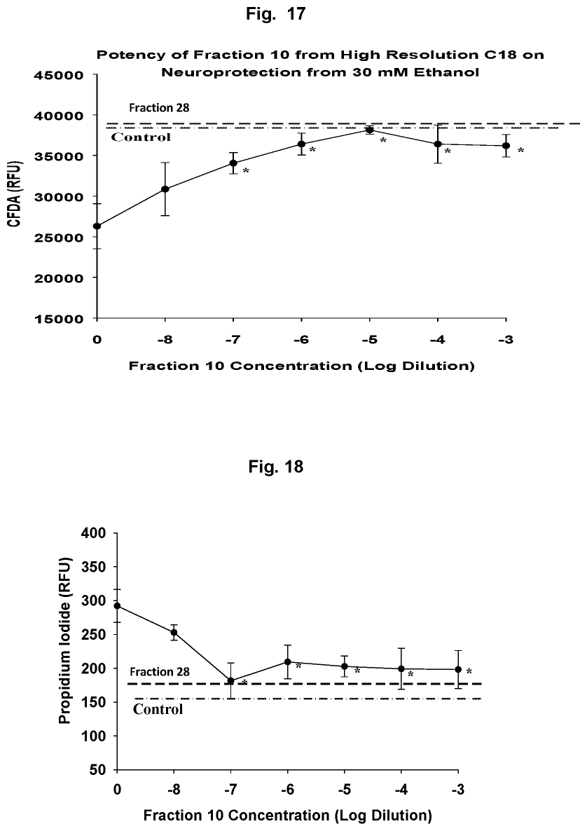

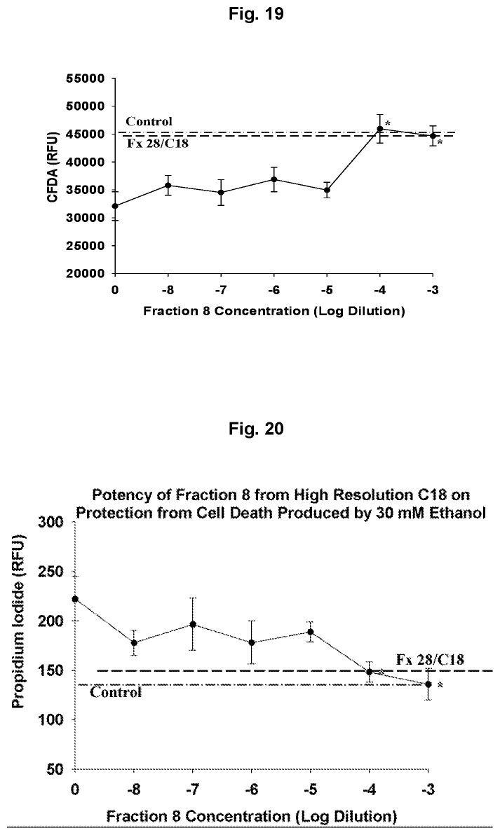

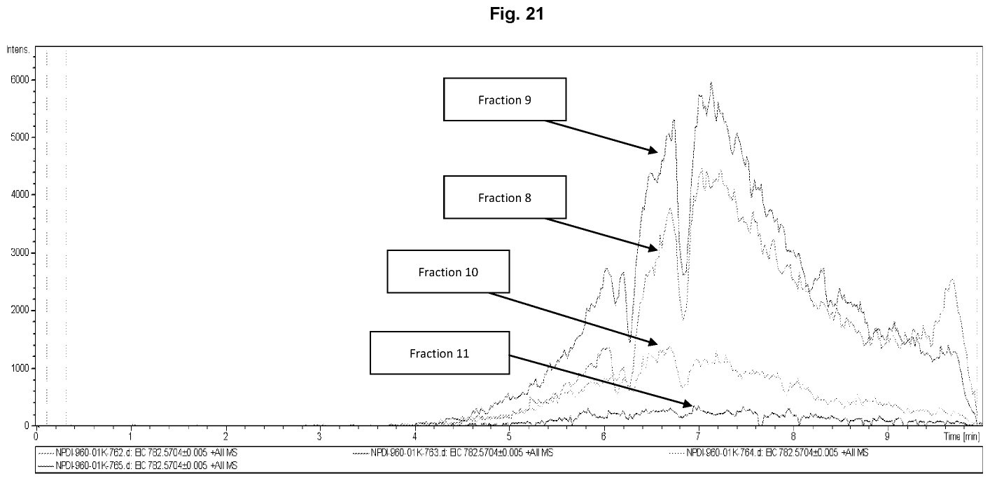

Fractions 9 and 10 were tested in a dose-response experiment for neuroprotective activity. As shown in FIG. 15 and FIG. 17, both fractions maintained neuroprotective activity down to a 10⁷-fold dilution. Both fractions 9 and 10 protected cells from death due to ethanol exposure down to a 10⁷-fold dilution as depicted in FIG. 16 and FIG. 18. It appeared that the activity was divided between the two fractions, and based on these results it appears that most of the neuroprotective activity of the crude hexane-soluble material from the H. lupulus fraction was concentrated into fractions 9 and 10. Fraction 8 was less potent than fractions 9 and 10, but did possess some neuroprotective activity (FIGS. 19 and 20).

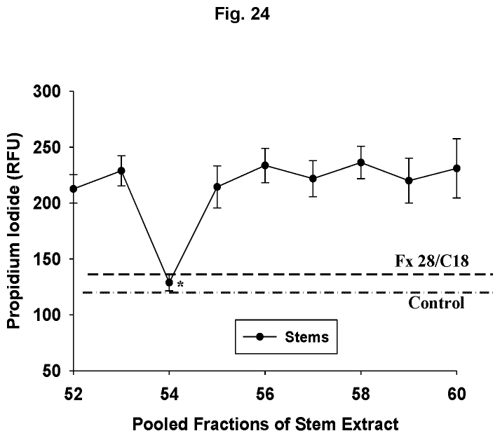

FIGS. 23-24 show the testing results of the fractions from H. lupulus stems of Example 3. The pool of fractions 18-21 (sample 54) was found to improve neuronal viability after exposure to ethanol as seen in FIG. 23, and also to prevent cell death as seen in FIG. 24.

Procedure for the Analysis of Fractions by High Resolution Mass Spectrometry.

Fractions from Examples 3 and 4 exhibiting neuroprotective activity were analyzed by high resolution mass spectrometry. Analytical C₁₈ HPLC fractions 9 and 10, exhibiting the most neuroprotective activity, along with fractions 8 and 11 and pooled fractions 18-21 from H. lupulus stems were analyzed. The analysis was performed on an Agilent 1200 Rapid Resolution HPLC interfaced to a Bruker maxis mass spectrometer. A Zorbax SD-C8 column (2.1 mm×30 mm, 3.5 m particle size, 0.3 mL/min) was used for the HPLC elution. Solvent A was 90:10 water:acetonitrile with 13 mM ammonium formate and 0.01% trifluoroacetic acid modifiers. Solvent B was 10:90 water:acetonitrile with 13 mM ammonium formate and 0.01% trifluoroacetic acid modifiers. A binary gradient was used for the elution. The gradient was as follows: 10%-100% B, 6 minutes; hold at 100% B for 2 minutes; return to original conditions over 0.1 minutes, re-equilibrate for 1.9 minutes. The mass spectrometer used positive mode electrospray ionization. The ion source parameters were: 4 kV capillary voltage, drying gas flow of 11 L/min at 200° C., and nebulizer pressure of 2.8 bar. Mass calibration was performed based on detection of sodium trifluoroacetate cluster ions.

Results of High Resolution Mass Spectrometry.

Analysis of analytical C₁₈ HPLC fractions 8-11 detected the presence of what were several unsaturated phosphatidylcholine lipid esters in these fractions. From fraction 9, ions were detected at m/z 782.5704 (calculated for C₄₄H₈₁NO₈P, 782.5699, Δ 0.6 ppm) and m/z 758.5701 (calculated for C₄₂H₈₁NO₈P, 758.5699, Δ 0.2 ppm). From fraction 10, those same ions were also detected, along with an ion at m/z 784.5857 (calculated for C₄₄H₈₃NO₈P, 784.5856, Δ 0.1 ppm). As shown in FIG. 21, the ion at m/z 782.5704 was found at various levels in fractions 8-10. Additionally, other phosphatidylcholine lipid esters were detected as being present in chromatography fractions from Humulus lupulus.

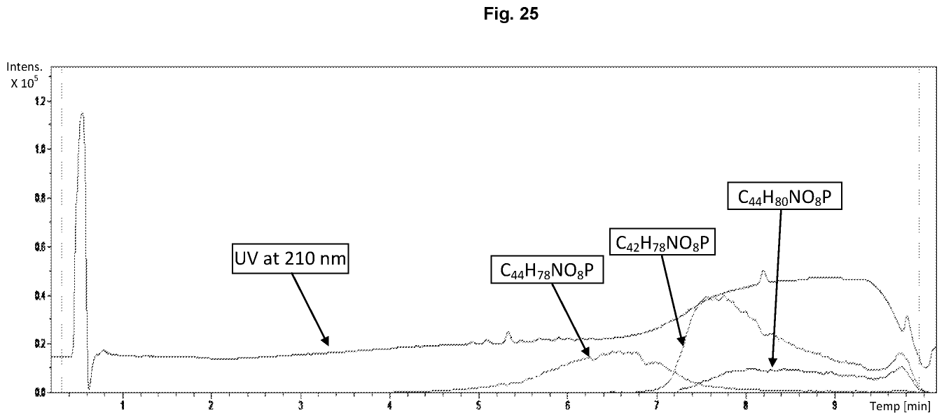

Analysis of pooled fractions 18-21 from H. lupulus stems detected the presence of phosphatidylcholine lipid esters with unsaturated lipid sidechains. As shown in FIG. 25, selective ion monitoring revealed the presence of ions corresponding to phosphatidylcholine lipid esters. Signals detected were at [M+H]=780.5540 (calculated for C₄₄H₇₈NO₈P, 780.5543, Δ 0.4 ppm); [M+H]⁺=756.5540 (calculated for C₄₂H₇₈NO₈P, 756.5543, Δ 0.4 ppm); and [M+H]⁺=782.5690 (calculated for C₄₂H₇₈NO₈P, 756.5599, Δ 1.2 ppm). The detected formula C₄₄H₇₈NO₈P could correspond to phosphatidylcholine esters with one 18:2 and one 18:3 lipid sidechain, such as 1-linolenoyl-2-linoleoyl-sn-glycero-3-phosphocholine or 1-linoleoyl-2-linolenoyl-sn-glycero-3-phosphocholine. Therefore, the neuroprotective effects found in the hexane extract of the stems of Humulus lupulus appear to correlate to the presence of phosphatidylcholine lipid esters with unsaturated lipid sidechains.

Table 2 shows the phosphatidylcholine structures from Humulus lupulus that are found in the chromatography fractions of Examples 1-4, exhibiting high potency neuroprotection from oxidative stress in hippocampal cultures:

Table 2: Exemplary compounds of the disclosure with neuroprotective activity in hippocampal cultures found in Humulus lupulus.

| | Chemical | Observed (m/z) | Calculated (m/z) |
|---|---|---|---|
| PC species | composition | [M + H]⁺ | [M + H]⁺ |
| PC 18:3-18:2 | C₄₄H₇₈NO₈P | 780.5540 | 780.5543 |
| PC 18:2-18:3 | C₄₄H₇₈NO₈P | 780.5540 | 780.5543 |
| PC 18:3-18:1 | C₄₄H₈₂NO₈P | 784.5857 | 784.5856 |
| PC 20:4-16:0 | C₄₄H₈₀NO₈P | 782.5704 | 782.5699 |

*PC = phosphatidylcholine

Results for Exemplary Commercially Available PCs of the Invention and Comparative Compounds.

Table 3 describes the potencies of the various embodiments of the inventions and comparative compounds as identified with the two assays: neuronal viability assay (CFDA—carboxyfluorescein diacetate) and cell death assay (PI— propidium iodide). The assays were conducted in rat dissociated hippocampal cultures during 5 hour test period with co-treatment with 30 mM ethanol to produce neurotoxicity and cell death as described in the Process section above.

TABLE 3
Neuroprotective activity for exemplary commercially available
PCs of the invention and comparative compounds.

| | | | CFDA | | Propidium Iodide | |
|---|---|---|---|---|---|---|
| PC Source | PC- sn-1 | PC- sn-2 | (EC50 + SE) | | (IC50 + SE) | |
| PC Avanti | 16:0 | 20:4 | 3079 ± 1886 | pM | 3500 ± 980 | pM |
| PC Avanti | 20:4 | 16:0 | 0.66 ± 0.32 | pM | 0.44 ± 0.02 | pM |
| PC Avanti | 20:4ω6* | 20:4 | 1.0 ± 0.08 | pM | 1.2 ± 0.12 | pM |
| PC Avanti | 18:2ω6 | 18:2 | 67 ± 22 | pM | 44 ± 15 | pM |
| PC Avanti | 22:6ω3** | 22:6 | 283 ± 46 | pM | 210 ± 53 | pM |
| PC Avanti | 18:3oo3 | 18:3 | 202 ± 22 | pM | 204 ± 88 | pM |

| | | | | |
|---|---|---|---|---|
| PC Avanti | 18:1ω9 | 18:1 | Inactive | Inactive |
| PC Avanti | 18:0 | 18:2 | Inactive | Inactive |
| PC Avanti | 16:0 | 18:2 | Inactive | Inactive |
| PC Avanti | 18:0 | 18:1 | Inactive | Inactive |
| PE Avanti | 18:2ω6 | 18:2 | Inactive | Inactive |
| PA Avanti | 18:2ω6 | 18:2 | Inactive | Inactive |

*20:4ω6 is an elongation and desaturation product of 18:2ω6
**22:6ω3 is an elongation and desaturation product of 18:3ω3
PC = phosphatidylcholine
PE = phosphatidylethanolamine
PA = phosphatidic acid

Custom synthesized by Avanti Polar Lipids.
Specifications of the synthesized PC 20:4-16:0 was as follows: Mass spectrometry molecular weight was 781.1, GC FAME was 99.4% pure, UV oxidation was 0.04% and 13C NMR data indicated no acyl migration detected in the final PC product.

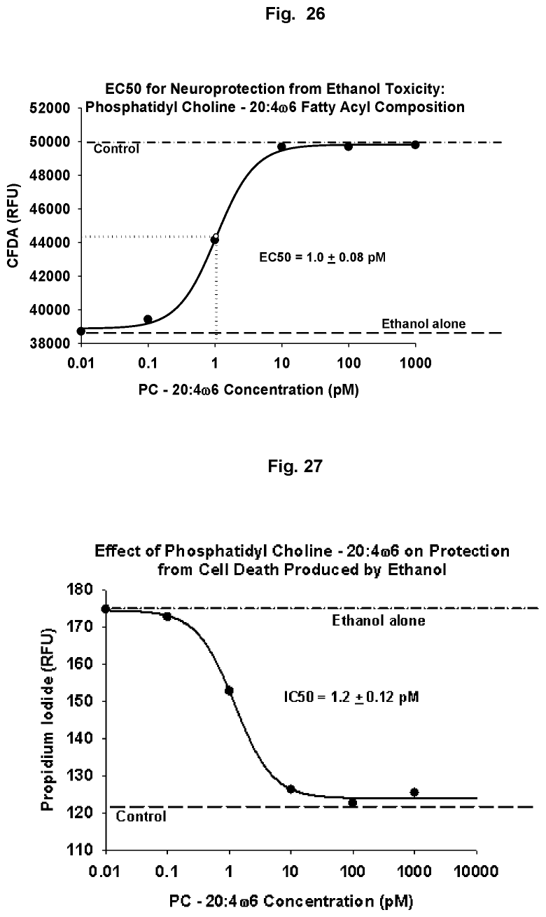

FIGS. 26 and 27 show the results of preferred embodiment 1, 2-diarachidonyl-phosphatidylcholine. FIG. 26 shows phosphatidylcholine with di-arachidonic acid in neuronal viability assay with an EC50 of 1.0±0.08 μM. FIG. 27 shows phosphatidylcholine with di-arachidonic acid in cell death assay with IC50 of 1.2±0.12 μM.

The exemplary formulations A1-A4 of Example 5 were tested alone and with another active agent. Cannabidiol was chosen as the candidate active agent for its neuroprotective effect.

TABLE 4
Neuroprotective activity for exemplary formulations A1-A4 of natural
compositions comprising PCs of the invention and comparative natural
phospholipid compositions alone or with cannabidiol.

| | PC- | PC- | CFDA | | Propidium Iodide | |
|---|---|---|---|---|---|---|
| PC Source | sn-1 | sn-2 | (EC50 + SE) | | (IC50 + SE) | |
| PC A1 alone | Soy | Soy | 53 ± 23 | pM | 160 ± 80 | pM |
| PC A1 CBD | Soy | Soy | 57 ± 21 | pM | 205 ± 3 | pM |
| PC A2 alone | Egg | Egg | Inactive | | Inactive | |
| PC A2 CBD | Egg | Egg | 1600 | nM | 1100 | nM |
| No PC A3 | none | none | Inactive | | Inactive | |
| No PC A3 CBD | none | none | Inactive | | Inactive | |
| PC A4 alone | Soy | Soy | 74 ± 21 | pM | 104 ± 31 | pM |
| PC A4 CBD | Soy | Soy | 85 ± 14 | pM | 118 ± 37 | pM |

PC = phosphatidylcholine
CBD = cannabidiol

The results of table 4 confirm the neuroprotective effect of PCs of the invention with PUFAs in the sn-1, and preferably in both the sn-1 and sn-2 positions. As described in literature, egg yolk lecithin comprises primarily of phospholipids with saturated sn-1 fatty acyl chains. (Cohen and Care, J Lipid Res. 32:1291, 1991.) In contrast, soybean lecithin comprises both saturated and unsaturated sn-1 fatty acyl chains. (Id.) Moreover, the PUFAs 18:2 and 18:3 make up over 52% by weight of the fatty acyl chains at the sn-1 position and over 76% of the fatty acyl chains at the sn-2 position. (Yamamoto et al., J. Oleo Sci. 63:1275, 2014.)

While the invention has been described in detail and with reference to specific examples thereof, it will be apparent to one skilled in the art that various changes and modifications can be made therein without departing from the spirit and scope thereof.

### REFERENCES

1. Brenneman, D E, Petkanas D, Kinney W A. (2018). Pharmacological comparison of cannabidiol and KLS-13019. J. Mol. Neurosci. 66:
2. Sugiura Y, Konishi Y, Zaima N, Kajihara S, Nakanishi H, Taguchi R, Setou M. (2009). Visualization of the cell-specific distribution of PUFA-containing phosphatidylcholines in mouse brain by imaging spectrometry. J. Lipid Res. 50: 1776-1788.
3. Yamashita A, Sugiura T, Waku K. (1997). Acyltransferases and transacylases involved in fatty acid remodeling of phospholipids and metabolism of bioactive lipids in mammalian cells. J. Biochem. 122:1-16/
4. Piomelli D, Astarita G, Rapaka R. (2007). A neuroscientist's guide to lipidomics. Nat. Rev Neurosci. 8:743-754.
5. Cao Q, Mak K M, Lieber C S (2002). DLPC decreases TGG-31-induced collagen mRNA by inhibiting p38 MAPK in hepatic stellate cells. Am J Physiol Gastrointest Liver Physiol 283:G1051-G1061.
6. Cao Q, Mak K M Lieber C S (2002). Dilinoleoylphosphatidycholine decreases acetaldehyde-induced TNF-alpha generation in Kupffer cells of ethanol-fed rats. Biochem Biophys Res Commun 299:459-464.
7. Kafrawy O, Zerouga M, Stillwell W, Jesnski U. (1998). Docosahexaenoic acid in phosphatidylcholine mediates cytotoxicity more effectively than other omega-3 and omega-6 fatty acids. Cancer Lett 132:23-29.
8. Lieber C S (2005). Pathogenesis and treatment of alcoholic liver disease: progress over the lasts 50 year. Rocz Akad Med Bialymst 50: 7-20.

1. A pharmaceutical composition comprising at least 0.5 pM of a phosphatidylcholine (PC), wherein the PC comprises a first polyunsaturated fatty acid (PUFA) in an sn-1 position thereof and a second PUFA in an sn-2 position thereof, wherein the first PUFA comprises a first fatty acyl chain with at least 16 carbons and the second PUFA comprises a second fatty acyl chain with at least 16 carbons.

2. The pharmaceutical composition of claim 1, comprising 0.5 pM to 1 nM of the PC.

3. The pharmaceutical composition of claim 1, wherein the first PUFA and the second PUFA comprise an omega-3 (ω3) or an omega-6 (ω6) fatty acyl chain.

4. The pharmaceutical composition of claim 1, wherein the first PUFA and the second PUFA comprise a fatty acyl chain with 18 to 24 carbons.

5. The pharmaceutical composition of claim 1, wherein the first PUFA and the second PUFA are the same.

6. The pharmaceutical composition of claim 1, wherein the first PUFA and the second PUFA are different.

7. The pharmaceutical composition of claim 1, wherein the first PUFA is a member selected from the group consisting of 18:2ω6, 18:3ω3, 20:4ω6, and 22:6ω3.

8. The pharmaceutical composition of claim 1, wherein the second PUFA is a member selected from the group consisting of 18:2ω6, 18:3ω3, 20:4ω6, and 22:6ω3.

9. The pharmaceutical composition of claim 1, wherein the first PUFA is 20:4 and a fatty acyl chain in the sn-2 position is 16:0, 18:0 or 18:1.

10. The pharmaceutical composition of claim 1, wherein the first PUFA is 20:4ω6.

11. The pharmaceutical composition of claim 1, wherein the PC is a component of a natural extract.

12. The pharmaceutical composition of claim 11, wherein the natural extract is an extract of Humulus lupulus.

13. The pharmaceutical composition of claim 12, wherein the natural extract comprises at least one of the following PC species:

| | Chemical | Observed (m/z) | Calculated (m/z) |
|---|---|---|---|
| PC species | Composition | [M + H]⁺ | [M + H]⁺ |
| PC 18:3-18:2 | C₄₄H₇₈NO₈P | 780.5540 | 780.5543 |
| PC 18:2-18:3 | C₄₄H₇₈NO₈P | 780.5540 | 780.5543 |
| PC 18:3-18:1 | C₄₄H₈₂NO₈P | 784.5857 | 784.5856 |
| PC 20:4-16:0 | C₄₄H₈₀NO₈P | 782.5704 | 782.5699 |

alone or in combination in an amount greater than 50% of the natural extract.

14. The pharmaceutical composition of claim 11, wherein the natural extract is soy lecithin.

15. The pharmaceutical composition of claim 14, wherein the soy lecithin comprises phosphatidylcholine with a polyunsaturation of the fatty acyl composition of at least 63%.

16. The pharmaceutical composition of claim 1, which further comprises an excipient.

17. The pharmaceutical composition of claim 1, further comprising an additional active agent in addition to the PC.

18. The pharmaceutical composition of claim 17, wherein the additional active agent is cannabidiol (CBD).

19. The pharmaceutical composition of claim 1, wherein the PC is a membrane component of a liposome.

20. The pharmaceutical composition of claim 1, which is effective to treat or prevent oxidative stress in a neuronal tissue.

21. A method of treating or preventing oxidative stress in a neuronal tissue, said method comprising administering to a subject in need thereof the pharmaceutical composition of claim 1.

22. The method of claim 21, wherein the neuronal tissue comprises tissues and cells of the central nervous system.

23. The pharmaceutical composition of claim 1, wherein the PC comprises at least one of 1,2-dilinoleoyl-phosphatidylcholine, 1,2-dilinolenoyl-phosphatidylcholine, 1,2-diarachidonyl-phosphatidylcholine, 1-linolenoyl-2-linoleoyl-phosphatidylcholine, 1-linoleoyl-2-linolenoyl-phosphatidylcholine, 1-linolenoyl-2-oleoyl-phosphatidylcholine and 1-arachidonyl-2-palmitoyl-phosphatidylcholine.
